(19) United States
(12) Patent Application Publication
(10) Pub. No.: US 2022/0233449 A1
(43) Pub. Date: Jul. 28, 2022

(54) SOLID DISPERSION AND PREPARATION METHOD THEREFOR

(71) Applicant: Jiangsu Hengrui Medicine Co., Ltd., Lianyungang, Jiangsu (CN)

(72) Inventors: Xianqiang Zhou, Lianyungang, Jiangsu (CN); Zhenxing Du, Lianyungang, Jiangsu (CN); Jie Wang, Lianyungang, Jiangsu (CN)

(21) Appl. No.: 17/615,152

(22) PCT Filed: May 29, 2020
(86) PCT No.: PCT/CN2020/093206

§ 371 Date: Nov. 30, 2021

(30) Foreign Application Priority Data
May 31, 2019 (CN) 201910468254.9

(51) Int. Cl. A61K 9/14; A61K 31/5025

(52) U.S. Cl. CPC A61K 9/146; A61K 31/5025

(57) ABSTRACT

Solid dispersion and a preparation method therefor. In a specific embodiment, the solid dispersion contains an active ingredient (R)-4-amino-1-(1-(but-2-ynyl)pyrrolidin-3-yl))-3-(4-(2,6-difluorophenoxy)phenyl)-1,6-dihydro-7H-pyrrolo[2,3-d]pyridazin-7-one or a salt thereof, and a carrier material, and the pH value is adjusted; employing a method that adds an appropriate amount of acid effectively inhibits an emulsification phenomenon in a reverse solvent process, thereby obtaining solid dispersion having a moderate particle size and uniform content.

### TECHNICAL FIELD

The present disclosure belongs to the field of pharmaceutical preparations, and specifically relates to a solid dispersion, a method for preparing the same and a use thereof.

### BACKGROUND

The compound of formula I (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one is a BTK inhibitor with good target specificity and high selectivity for kinase. It can inhibit BTK phosphorylation and down-regulate BCR signal transduction pathway, thereby selectively inhibiting the proliferation and migration of B cell tumor. Recent clinical trials have shown that it has excellent pharmacodynamic activity,

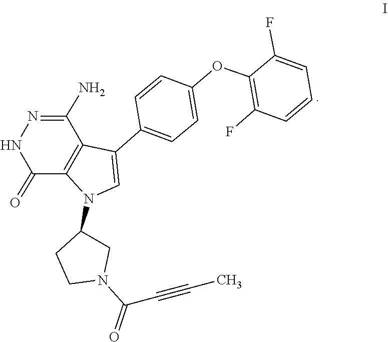 [SMILES: CC#CC(=O)N1CC[C@@H](n2cc(-c3ccc(Oc4c(F)cccc4F)cc3)c3c(N)n[nH]c(=O)c32)C1]

The compound of formula I is an active substance with low solubility, and its druggability needs to be studied in depth and solved by pharmaceutical researchers. WO2019007317 discloses a solid dispersion preparation containing the compound of formula I, which applies solid dispersion technology to solve the problem of dissolution after the compound is formulated as a drug. The methods for preparing solid dispersion include melting method, solvent method, solvent-melting method, solvent-spray drying method or grinding method. The anti-solvent method is the mildest method for preparing solid dispersion, which avoids subjecting active substance to high temperature, and is suitable for various heat-labile or volatile drugs, and is easy to conduct.

However, during the preparation of solid dispersion, especially when the anti-solvent method is used, emulsification often occurs, which will affect the preparation of solid dispersion, resulting in uneven content of active ingredient between batches, thereby affecting the effectiveness and safety.

Common demulsification methods include static method, high-voltage electric field method, chemical demulsification method (such as salt fractionation method, coagulation method, salt fractionation-coagulation method), centrifugal method, ultrafiltration method and vacuum separation method. Each demulsification method has its own advantages, while accompanied by its own disadvantages. The chemical demulsification method such as the salt fractionation method needs to introduce an inorganic salt as an additional chemical reagent into the emulsification system, thereby increasing the cost of subsequent processing. At the same time, the selection of demulsification method also needs to consider the characteristics of the product itself.

### SUMMARY OF THE INVENTION

The present disclosure provides a method for preparing a solid dispersion, comprising the steps of dissolving a carrier material and an active ingredient in a good solvent to obtain a solution, adding the resulting solution to a poor solvent, and adjusting the pH to 1.0 to 6.0; or, comprising the steps of dissolving a carrier material and an active ingredient in a good solvent to obtain a solution, and adding the resulting solution to a poor solvent, wherein the pH of the poor solvent is 1.0 to 6.0.

In alternative embodiments, the pH value can be 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0 or any value between any two values, and preferably the pH is 2.0 to 4.0.

Controlling the pH of the poor solvent or the crystallization solution to be less than 6.0 can effectively avoid the aforementioned emulsification problem during the anti-solvent method. There is no need to conduct additional demulsification methods such as salt fractionation method and coagulation method, which facilitates subsequent filtration or washing process, and provides samples with even active ingredient content between batches.

Furthermore, the method of the present disclosure also comprises a step of filtration, washing or drying.

In some embodiments, the good solvent is at least one selected from the group consisting of dimethyl sulfoxide, N,N-dimethylformamide, N,N-dimethylacetamide, acetone, ethanol, tetrahydrofuran and methanol, and preferably N,N-dimethylformamide or N,N-dimethylacetamide; and the poor solvent is at least one selected from the group consisting of diethyl ether, n-hexane, petroleum ether and water, such as a mixed solution of water with one or more of ether, n-hexane and petroleum ether.

In some embodiments, the carrier material and the active ingredient or the pharmaceutically acceptable salt thereof are dissolved in the good solvent of N,N-dimethylformamide, and the resulting solution is added to the poor solvent of water, wherein the pH of the poor solvent of water is 1.0 to 6.0.

In some embodiments, the carrier material and the active ingredient or the pharmaceutically acceptable salt thereof are dissolved in the good solvent of N,N-dimethylacetamide, and the resulting solution is added to the poor solvent of water, wherein the pH of the poor solvent of water is 1.0 to 6.0.

In some embodiments, the carrier material and the active ingredient or the pharmaceutically acceptable salt thereof are dissolved in the good solvent of dimethyl sulfoxide, and the resulting solution is added to the poor solvent of water, wherein the pH of the poor solvent of water is 1.0 to 6.0.

Furthermore, the reagent used to adjust pH is at least one selected from the group consisting of hydrochloric acid, sulfuric acid, acetic acid and phosphoric acid, and preferably sulfuric acid or hydrochloric acid.

In another aspect, the rate of the dropwise addition (that is, the addition of the good solvent to the poor solvent) will also affect the quality of crystallization to a certain extent, such as the size and uniformity of the precipitated solid particles.

In some embodiments, the rate of the dropwise addition (the addition of the good solvent to the poor solvent) can be 1 to 2500 g/min, such as 1 to 2250 g/min, 1 to 2000 g/min. The rate of the dropwise addition can be adjusted according to the needs of preparation.

In other embodiments, the addition to the poor solvent of the present disclosure is accompanied by a stirring process, the stirring rate is selected from the group consisting of 20 to 1000 rpm, non-limiting examples include 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 rpm or any value between any two values, and preferably 100 to 600 rpm.

The method for preparing a solid dispersion of the present disclosure can be applied to any insoluble solids. In alternative embodiments, the active ingredient is (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one or the pharmaceutically acceptable salt thereof.

Furthermore, the carrier material is at least one selected from the group consisting of hydroxypropyl methylcellulose succinate, polyvinylpyrrolidone, hydroxypropyl methylcellulose phthalate and polyvinylpyrrolidone.

In some embodiments, the carrier material hydroxypropyl methylcellulose acetate succinate and the active ingredient are dissolved in the good solvent of N,N-dimethylformamide, and the resulting solution is added to the poor solvent of water, wherein the pH of the poor solvent of water=1.0 to 6.0.

In some embodiments, the carrier material of hydroxypropyl methylcellulose acetate succinate and the active ingredient are dissolved in the good solvent N,N-dimethylacetamide, and the resulting solution is added to the poor solvent of water, wherein the pH of the poor solvent of water is 1.0 to 6.0.

In some embodiments, the carrier material of hydroxypropyl methylcellulose acetate succinate and the active ingredient are dissolved in the good solvent of dimethyl sulfoxide, and the resulting solution is added to the poor solvent of water, wherein the pH of the poor solvent of water is 1.0 to 6.0.

In some embodiments, the carrier material of polyvinylpyrrolidone and the active ingredient are dissolved in the good solvent of N,N-dimethylformamide, and the resulting solution is added to the poor solvent of water, wherein the pH of the poor solvent of water is 1.0 to 6.0.

In some embodiments, the carrier material of polyvinylpyrrolidone and the active ingredient are dissolved in the good solvent of N,N-dimethylacetamide, and the resulting solution is added to the poor solvent of water, wherein the pH of the poor solvent of water is 1.0 to 6.0.

In some embodiments, the carrier material of polyvinylpyrrolidone and the active ingredient are dissolved in the good solvent of dimethyl sulfoxide, and the resulting solution is added to the poor solvent of water, wherein the pH of the poor solvent of water is 1.0 to 6.0.

In some embodiments, the carrier material of hydroxypropyl methylcellulose phthalate and the active ingredient are dissolved in the good solvent of N,N-dimethylformamide, and the resulting solution is added to the poor solvent of water, wherein the pH of the poor solvent of water is 1.0 to 6.0.

In some embodiments, the carrier material of hydroxypropyl methylcellulose phthalate and the active ingredient are dissolved in the good solvent of N,N-dimethylacetamide, and the resulting solution is added to the poor solvent of water, wherein the pH of the poor solvent of water is 1.0 to 6.0.

In some embodiments, the carrier material of hydroxypropyl methylcellulose phthalate and the active ingredient are dissolved in the good solvent of dimethyl sulfoxide, and the resulting solution is added to the poor solvent of water, wherein the pH of the poor solvent of water is 1.0 to 6.0.

In some embodiments, the carrier material of hydroxypropyl methylcellulose acetate succinate and the active ingredient are dissolved in the good solvent of N,N-dimethylformamide, the resulting solution is added to the poor solvent of water, and the pH is adjusted to 1.0 to 6.0.

In some embodiments, the carrier material of hydroxypropyl methylcellulose acetate succinate and the active ingredient are dissolved in the good solvent of N,N-dimethylacetamide, the resulting solution is added to the poor solvent of water, and the pH is adjusted to 1.0 to 6.0.

In some embodiments, the carrier material of hydroxypropyl methylcellulose acetate succinate and the active ingredient are dissolved in the good solvent of dimethyl sulfoxide, the resulting solution is added to the poor solvent of water, and the pH is adjusted to 1.0 to 6.0.

In some embodiments, the carrier material of polyvinylpyrrolidone and the active ingredient are dissolved in the good solvent of N,N-dimethylformamide, the resulting solution is added to the poor solvent of water, and the pH is adjusted to 1.0 to 6.0.

In some embodiments, the carrier material of polyvinylpyrrolidone and the active ingredient are dissolved in the good solvent of N,N-dimethylacetamide, the resulting solution is added to the poor solvent of water, and the pH is adjusted to 1.0 to 6.0.

In some embodiments, the carrier material of polyvinylpyrrolidone and the active ingredient are dissolved in the good solvent of dimethyl sulfoxide, the resulting solution is added to the poor solvent of water, and the pH is adjusted to 1.0 to 6.0.

In some embodiments, the carrier material of hydroxypropyl methylcellulose phthalate and the active ingredient are dissolved in the good solvent of N,N-dimethylformamide, the resulting solution is added to the poor solvent of water, and the pH is adjusted to 1.0 to 6.0.

In some embodiments, the carrier material of hydroxypropyl methylcellulose phthalate and the active ingredient are dissolved in the good solvent of N,N-dimethylacetamide, the resulting solution is added to the poor solvent of water, and the pH is adjusted to 1.0 to 6.0.

In some embodiments, the carrier material of hydroxypropyl methylcellulose phthalate and the active ingredient are dissolved in the good solvent of dimethyl sulfoxide, the resulting solution is added to the poor solvent of water, and the pH is adjusted to 1.0 to 6.0.

In some embodiments, the method of the present disclosure comprises the following steps of:

a) dissolving the carrier material and the active ingredient (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one or the pharmaceutically acceptable salt thereof in a good solvent, wherein the good solvent is at least one selected from the group consisting of dimethyl sulfoxide, N,N-dimethylformamide, N,N-dimethylacetamide, acetone, ethanol, tetrahydrofuran and methanol,

b) adding the solution obtained in step a) to a poor solvent, and adjusting the pH to 1.0 to 6.0, wherein the poor solvent is at least one selected from the group consisting of diethyl ether, n-hexane, petroleum ether and water.

In other embodiments, the method of the present disclosure comprises the following steps of:

a) dissolving the carrier material and the active ingredient (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one or the pharmaceutically acceptable salt thereof in a good solvent, wherein the good solvent is at least one selected from the group consisting of dimethyl sulfoxide, N,N-dimethylformamide, N,N-dimethylacetamide, acetone, ethanol, tetrahydrofuran and methanol,

b) adding the solution obtained in step a) to a poor solvent, wherein the poor solvent is selected from the group consisting of water and a mixed solution of water with one or more of ether, n-hexane and petroleum ether, and the pH of the poor solvent is 1.0 to 6.0.

Furthermore, the method also comprises a step of filtration, washing or drying.

In other embodiments, the method of the present disclosure also comprises a step of spray drying.

In other embodiments, the particle size D90 of the solid dispersion is 50 μm to 2000 μm.

The particle size D90 of the solid dispersion provided in some embodiments can be selected from the group consisting of 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm, 810 μm, 820 μm, 830 μm, 840 μm, 850 μm, 860 μm, 870 μm, 880 μm, 890 μm, 900 μm, 910 μm, 920 μm, 930 μm, 940 μm, 950 μm, 960 μm, 970 μm, 980 μm, 990 μm, 1000 μm, 1010 μm, 1020 μm, 1030 μm, 1040 μm, 1050 μm, 1060 μm, 1070 μm, 1080 μm, 1090 μm, 1100 μm, 1110 μm, 1120 μm, 1130 μm, 1140 μm, 1150 μm, 1160 μm, 1170 μm, 1180 μm, 1190 μm, 1200 μm, 1210 μm, 1220 μm, 1230 μm, 1240 μm, 1250 μm, 1260 μm, 1270 μm, 1280 μm, 1290 μm, 1300 μm, 1310 μm, 1320 μm, 1330 μm, 1340 μm, 1350 μm, 1360 μm, 1370 μm, 1380 μm, 1390 μm, 1400 μm, 1410 μm, 1420 μm, 1430 μm, 1440 μm, 1450 μm, 1460 μm, 1470 μm, 1480 μm, 1490 μm, 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710 μm, 1720 μm, 1730 μm, 1740 μm, 1750 μm, 1760 μm, 1770 μm, 1780 μm, 1790 μm, 1800 μm, 1810 μm, 1820 μm, 1830 μm, 1840 μm, 1850 μm, 1860 μm, 1870 μm, 1880 μm, 1890 μm, 1900 μm, 1910 μm, 1920 μm, 1930 μm, 1940 μm, 1950 μm, 1960 μm, 1970 μm, 1980 μm, 1990 μm, 2000 and any value between any two values, preferably 100 μm to 1500 μm, and more preferably 100 μm to 1000 μm.

Furthermore, the particle size D50 of the solid dispersion provided in some embodiments is 20 μm to 500 μm. Non-limiting examples include 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm or any value between any two values, and preferably 20 μm to 200 μm.

Furthermore, the particle size D10 of the solid dispersion provided in some embodiments is 1 μm to 100 μm. Non-limiting examples include 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm or any value between any two values, and preferably 5 μm to 50 μm.

Furthermore, the particle size D [4,3] of the solid dispersion provided in some embodiments is 100 μm to 800 μm. Non-limiting examples include 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm or any value between any two values, and preferably 150 μm to 600 μm.

The particle size D90 of the solid dispersion provided in the most preferred embodiment is 100 μm to 1000 μm, the particle size D50 of the same is 20 μm to 200 μm, and the particle size D10 of the same is 1 μm to 100 μm.

The particle size of the solid dispersion of the present disclosure refers to the particle size of the solid dispersion in the suspension obtained after the particles precipitated from the solvent, or the particle size of the solid dispersion after removing the solvent and drying. In some embodiments, the particle size refers to the particle size of the solid dispersion in the suspension obtained after the particles precipitated from the solvent.

In some embodiments, the active ingredient is in non-crystalline form.

In another aspect, the solid dispersion (SD) refers to a dispersion system in solid form formed by highly dispersing a drug in a solid carrier. The carrier material provides a dispersion system for the active ingredient. The higher the content of the carrier material, the easier it is for the active ingredient to transform from crystalline to amorphous, and the higher the bioavailability of the corresponding solid dispersion. In view of the balance between drug loading and bioavailability, the weight ratio of the carrier material to the active ingredient of the present disclosure can be 0.5:1 to 4:1. In some embodiments, the weight ratio can be 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.2:1, 2.4:1, 2.6:1, 2.8:1, 3:1, 3.2:1, 3.4:1, 3.6:1, 3.8:1, 4:1 or any value between any two values, and preferably 0.8:1 to 3:1.

In the solid dispersion provided in some embodiments, the carrier material is at least one selected from the group consisting of hydroxypropyl methylcellulose acetate succinate, polyvinylpyrrolidone, hydroxypropyl methylcellulose phthalate and polyvinylpyrrolidone.

The solid dispersion provided in some preferred embodiments comprises polyvinylpyrrolidone and the active ingredient (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one or the pharmaceutically acceptable salt thereof.

Furthermore, the solid dispersion of the present disclosure consists of the active ingredient (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one or the pharmaceutically acceptable salt thereof and the carrier material.

The present disclosure also provides a solid dispersion prepared by the above method. The present disclosure also provides a solid dispersion comprising the active ingredient (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one or the pharmaceutically acceptable salt thereof and a carrier material, wherein the particle size D90 of the solid dispersion is 50 μm to 2000 μm.

The particle size D90 of the solid dispersion provided in some embodiments can be selected from the group consisting of 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm, 810 μm, 820 μm, 830 μm, 840 μm, 850 μm, 860 μm, 870 μm, 880 μm, 890 μm, 900 μm, 910 μm, 920 μm, 930 μm, 940 μm, 950 μm, 960 μm, 970 μm, 980 μm, 990 μm, 1000 μm, 1010 μm, 1020 μm, 1030 μm, 1040 μm, 1050 μm, 1060 μm, 1070 μm, 1080 μm, 1090 μm, 1100 μm, 1110 μm, 1120 μm, 1130 μm, 1140 μm, 1150 μm, 1160 μm, 1170 μm, 1180 μm, 1190 μm, 1200 μm, 1210 μm, 1220 μm, 1230 μm, 1240 μm, 1250 μm, 1260 μm, 1270 μm, 1280 μm, 1290 μm, 1300 μm, 1310 μm, 1320 μm, 1330 μm, 1340 μm, 1350 μm, 1360 μm, 1370 μm, 1380 μm, 1390 μm, 1400 μm, 1410 μm, 1420 μm, 1430 μm, 1440 μm, 1450 μm, 1460 μm, 1470 μm, 1480 μm, 1490 μm, 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710 μm, 1720 μm, 1730 μm, 1740 μm, 1750 μm, 1760 μm, 1770 μm, 1780 μm, 1790 μm, 1800 μm, 1810 μm, 1820 μm, 1830 μm, 1840 μm, 1850 μm, 1860 μm, 1870 μm, 1880 μm, 1890 μm, 1900 μm, 1910 μm, 1920 μm, 1930 μm, 1940 μm, 1950 μm, 1960 μm, 1970 μm, 1980 μm, 1990 μm, 2000 and any value between any two values, preferably 100 μm to 1500 μm, and more preferably 100 μm to 1000 μm.

Furthermore, the particle size D50 of the solid dispersion provided in some embodiments is 20 μm to 500 μm. Non-limiting examples include 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm or any value between any two values, and preferably 20 μm to 200 μm.

Furthermore, the particle size D10 of the solid dispersion provided in some embodiments is 1 μm to 100 μm. Non-limiting examples include 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm or any value between any two values, and preferably 5 μm to 50 μm.

Furthermore, the particle size D [4,3] of the solid dispersion provided in some embodiments is 100 μm to 800 μm. Non-limiting examples include 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm or any value between any two values, and preferably 150 μm to 600 μm.

The particle size D90 of the solid dispersion provided in the most preferred embodiment is 100 μm to 1000 μm, the particle size D50 of the same is 20 μm to 200 μm, and the particle size D10 of the same is 1 μm to 100 μm.

In some embodiments, the active ingredient is in non-crystalline form.

In view of the balance between drug loading and bioavailability, the weight ratio of the carrier material to the active ingredient of the present disclosure can be 0.5:1 to 4:1. In some embodiments, the weight ratio can be 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.2:1, 2.4:1, 2.6:1, 2.8:1, 3:1, 3.2:1, 3.4:1, 3.6:1, 3.8:1, 4:1 or any value between any two values, and preferably 0.8:1 to 3:1.

In the solid dispersion provided in some embodiments, the carrier material is at least one selected from the group consisting of hydroxypropyl methylcellulose acetate succinate, polyvinylpyrrolidone, hydroxypropyl methylcellulose phthalate and polyvinylpyrrolidone.

The solid dispersion provided in some preferred embodiments comprises polyvinylpyrrolidone and the active ingredient (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one or the pharmaceutically acceptable salt thereof.

Furthermore, the solid dispersion of the present disclosure is consisting of the active ingredient (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one or the pharmaceutically acceptable salt thereof and the carrier material.

In another aspect, the present disclosure provides a solid formulation comprising the above solid dispersion or the solid dispersion prepared by the above method and optionally a pharmaceutically acceptable excipient, wherein the excipient is at least one selected from the group consisting of disintegrant, filler, binder and lubricant. The solid formulation can be a tablet, pill, granule, capsule, or the like.

In some embodiments, the content of the active ingredient is 8 to 40% by weight, relative to the weight of the pharmaceutical composition. The content of the active ingredient can be 8, 9, 10, 11, 12, 13, 14, 15, 16, 17, 18, 19, 20, 21, 22, 23, 24, 25, 26, 27, 28, 29, 30, 35, 40% or any value between any two values, and preferably 15 to 25% by weight, relative to the weight of the pharmaceutical composition.

In other embodiments, the amount (weight or mass) of the active ingredient is 10 to 500 mg. The the amount (weight or mass) of the active ingredient can be 200 mg, 190 mg, 180 mg, 170 mg, 160 mg, 150 mg, 140 mg, 130 mg, 120 mg, 110 mg, 100 mg, 95 mg, 75 mg, 50 mg, 25 mg, 15 mg, 10 mg or any value between any two values, and preferably 200 mg, 100 mg or 25 mg.

The disintegrant of the present disclosure is known or determinable by those skilled in the art, and selected, but not limited to, at least one of croscarmellose sodium, crospovidone, sodium carboxymethyl starch, starch, pregelatinized starch and alginic acid.

Preferably, the disintegrant is present in an amount of 1 to 20% by weight, relative to the weight of the pharmaceutical composition. Non-limiting examples include 1.0, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20% or any value between any two values, and preferably 5 to 15% by weight, relative to the weight of the pharmaceutical composition.

The binder of the present disclosure is known or determinable by those skilled in the art, and includes, but not limited to, at least one of polyvinylpyrrolidone, starch, methyl cellulose, carboxy cellulose, hydroxypropyl cellulose, hydroxypropyl methyl cellulose and alginate, preferably at least one of polyvinylpyrrolidone (trade name K30) and hydroxypropyl cellulose. More preferably, the binder is present in an amount of 0.5 to 10% by weight, relative to the weight of the pharmaceutical composition. Non-limiting examples include 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10% or any value between any two values by weight, relative to the weight of the pharmaceutical composition.

The lubricant of the present disclosure is known or determinable by those skilled in the art, and includes, but not limited to, at least one of magnesium stearate, stearic acid, palmitic acid, calcium stearate, talc, carnauba wax and sodium stearyl fumarate. Preferably, the lubricant is present in an amount of 0.1 to 5% by weight, relative to the weight of the pharmaceutical composition. Non-limiting examples include 0.1, 0.2, 0.3, 0.4, 0.5, 0.6, 0.7, 0.8, 0.9%, 1%, 1.5%, 2%, 2.5%, 3%, 3.5%, 4%, 4.5, 5% or any value between any two values, and preferably 0.1 to 2% by weight, relative to the weight of the pharmaceutical composition.

In some embodiments, the solid formulation of the present disclosure comprises:

1) 10 mg to 500 mg of the active ingredient,

2) 5 to 15% by weight of the disintegrant,

3) 30 to 90% by weight of the filler,

4) 0.5 to 10% by weight of the binder,

5) 0.1 to 5% by weight of the lubricant.

Furthermore, the dissolution rate is determined according to the second method (paddle method) of the dissolution rate test described in general rule of volume IV of Chinese Pharmacopoeia 2015 Edition, using 0.15% aqueous solution of SDS as a dissolution medium, at 37±0.5° C., and at a paddle speed of 50 rpm. The dissolution rate (%) in 45 minutes of the active ingredient in the solid formulation of the present disclosure is 85% or greater, and can be greater than or equal to 85, 86, 87, 88, 89, 90, 91, 92, 93, 94, 95, 96, 97, 98, 99, 100%, and preferably 90% or greater. Furthermore, the dissolution rate (%) in 15 minutes of the active ingredient in the solid formulation is 70% or greater, and can be greater than or equal to 70, 71, 72, 73, 74, 75, 76, 77, 78, 79, 80, 81, 82, 83, 84, 85, 86, 87, 88, 89, 90, 91, 92, 93, 94, 95%. The solid formulation dissolves rapidly and completely, and has a good bioavailability. The preparation process of the solid formulation is simple, and suitable for large-scale production.

Furthermore, the present disclosure also provides a method for preparing the above solid formulation comprising the steps of pulverizing the solid dispersion, mixing well with the filler and/or disintegrant required for molding a pharmaceutical composition, adding the binder, subjecting to wet granulation or dry granulation, drying the resulting granules, screening by a sieve, milling, mixing well with the lubricant, and preparing into pills or granules or compressing into tablets or filling into capsules; or the solid dispersion can also be added directly into a capsule with suitable auxiliary materials or be compressed into tablets. The resulting granules or raw tablets or capsules can be further coated as needed.

The present disclosure also provides a use of the solid dispersion or solid formulation of the present disclosure in the preparation of a medicament for the treatment of conditions or diseases mediated by protein tyrosine kinase. In some embodiments, the condition or disease is a cancer or autoimmune disease. In some embodiments, the cancer is a B cell malignancy selected from the group consisting of chronic lymphocytic leukemia (CLL), mantle cell lymphoma (MCL), diffuse large B-cell lymphoma (DLBCL), multiple myeloma (MM), follicular lymphoma (FL), marginal zone lymphoma and Waldenstrom's macroglobulinemia (WM). In some embodiments, the autoimmune disease is rheumatoid arthritis or systemic lupus erythematosus.

The active ingredient (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one of the present disclosure can be formed into a pharmaceutically acceptable salt with an acid. The acid is known or determinable by those skilled in the art and includes, but is not limited to, hydrochloric acid, methanesulfonic acid, fumaric acid, trifluoroacetic acid and phosphoric acid.

The expression “relative to the weight of the pharmaceutical composition” of the present disclosure means that the calculation of the range of the amount of the active ingredient or other kinds of pharmaceutical auxiliary materials is based on the weight of the tablet core without a coating agent.

The “good solvent” and “poor solvent (weak solvent)” of the present disclosure are classified according to the solubility of the active ingredient. In general, poor solvent and good solvent are classified by the solubility of 20 mg/ml. If a solvent has a solubility of the active ingredient lower than about 20 mg/ml, then the solvent is a poor solvent. Correspondingly, if a solvent has a solubility of the active ingredient higher than about 20 mg/ml, then the solvent is a good solvent.

The term “D10” of the present disclosure refers to the corresponding particle size when the cumulative particle size distribution percentage of a sample reaches 10%. The term “D50” refers to the corresponding particle size when the cumulative particle size distribution percentage of a sample reaches 50%. The term “D90” refers to the corresponding particle size when the cumulative particle size distribution percentage of a sample reaches 90%. D[4,3] represents the “fourth moment/volume” average diameter, also known as the volume (or weight) average diameter. For those skilled in the art, there is a certain degree of error in particle size measurement. In general, plus or minus 10% are within a reasonable error range. D10, D50, D90 and D[4,3] have a certain degree of error variation depending on the context in which they are used, and the error variation does not exceed plus or minus 10%.

HPLC Detection Conditions of the Present Disclosure:

Octadecylsilane bonded silica is used as the filler (Waters Symmetry C18 column); 0.01 mol/L potassium dihydrogen phosphate buffer solution and acetonitrile are used as the mobile phase and eluent; the detection wavelength is 210 nm.

The pharmaceutical auxiliary materials and reagents, such as hydroxypropyl methylcellulose acetate succinate, are commercially available. (R)-4-Amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one (compound A) or a pharmaceutically acceptable salt thereof can be prepared according to the method described in Example 109 of WO2016007185.

### DESCRIPTION OF THE DRAWINGS

The above and other objects and features of the present disclosure will become apparent with reference to the following drawings, which respectively represent:

FIG. 1: SEM image of the sample of Example 3

FIG. 2: SEM image of the sample of Example 4

### DETAILED DESCRIPTION

The present disclosure will be further described in detail with reference to the following examples and experimental examples. These examples and experimental examples are for illustrative purposes only, and should not be considered as limiting the scope of the present disclosure.

### Example 1

1 g of (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one (abbreviated as compound A) and 1 g of hydroxypropyl methylcellulose acetate succinate (HPMC-AS) were added to 15 ml of dimethylacetamide, and stirred to dissolve. The resulting solution was added dropwise into 100 ml of water at a rate of 4 g/min or 2 g/min, and stirred for about 1 hour. The resulting suspension was filtered. Agglomeration was observed initially, filtration (or suction filtration) was difficult, and the suction filtrate appeared milky.

### Example 2

1 g of compound A and 1 g of hydroxypropyl methylcellulose acetate succinate (HPMC-AS) were added to 15 ml of dimethylacetamide, and stirred to dissolve. The resulting solution was added dropwise at a rate of 4 g/min into 100 ml of water with different pH (shown in Table 1), and stirred for about 1 hour. The resulting suspension was filtered to obtain solid. The observed phenomena are as follows:

TABLE 1

| | | | | | Active | |
|---|---|---|---|---|---|---|
| Experimental | | | Powder | | ingredient | |
| example | pH | Acid type | appearance | Phenomenon | content % | XRPD |
| 1 | 2 | 98% | Fluffy | Evenly dispersed, | 47.39% | Amorphous |
| | | sulfuric acid | | easy to filter, and | | |
| | | | | the filtrate was | | |
| | | | | clear | | |
| 2 | 2 | 85% | Fluffy | Evenly dispersed, | 50.88% | Amorphous |
| | | phosphoric | | easy to filter, and | | |
| | | acid | | the filtrate was | | |
| | | | | clear | | |
| 3 | 3 | Glacial | Fluffy | Evenly dispersed, | 47.25% | Amorphous |
| | | acetic acid | | easy to filter, and | | |
| | | | | the filtrate was | | |
| | | | | clear | | |
| 4 | 3 | 98% | Fluffy | Evenly dispersed, | 50.75% | Amorphous |
| | | sulfuric acid | | easy to filter, and | | |
| | | | | the filtrate was | | |
| | | | | clear | | |
| 5 | 3 | 36.5% | Tight | Evenly dispersed, | 46.35% | Amorphous |
| | | hydrochloric | | easy to filter, and | | |
| | | acid | | the filtrate was | | |
| | | | | clear | | |

Conclusion: The solid dispersion prepared with phosphoric acid or sulfuric acid has low residual ion content. For example, when using phosphoric acid, the phosphorus content in the obtained solid dispersion is about 20 μg/g; and when using sulfuric acid, the sulfur content in the obtained solid dispersion is 10 μg/g.

The samples obtained in Experimental Examples 1, 4 and 5 were placed at 93%, 60° C., 40° C./75% RH, 25° C./60% RH conditions respectively to investigate the physical and chemical stability. Data are shown as follows:

| Conditions | | | | |
|---|---|---|---|---|
| Humidity (%) | Temperature (°C) | 1 | 4 | 5 |
| | | Day 7 | | |
| 60 | 25 | 99.42% | 99.40% | 99.37% |
| 75 | 40 | 99.41% | 99.38% | 99.38% |
| 93 | / | 99.42% | 99.40% | 99.39% |
| / | 60 | 99.42% | 99.39% | 99.38% |
| | | Day 14 | | |
| 60 | 25 | 99.41% | 99.39% | 99.43% |
| 75 | 40 | 99.40% | 99.40% | 99.40% |
| 93 | / | 99.39% | 99.41% | 99.42% |
| / | 60 | 99.39% | 99.40% | 99.41% |

### Example 3

1 g of compound A and 1 g of HPMC-AS were dissolved in 15 ml of N,N-dimethylacetamide. The resulting solution was added dropwise at a rate of 4 g/min into 100 ml of water solution (adjusted to pH=2 with 36.5% hydrochloric acid), and stirred for 30 minutes. The resulting suspension was filtered, and the filter cake was rinsed with water. The resulting solid was dried overnight at 40° C., and subjected to SEM determination.

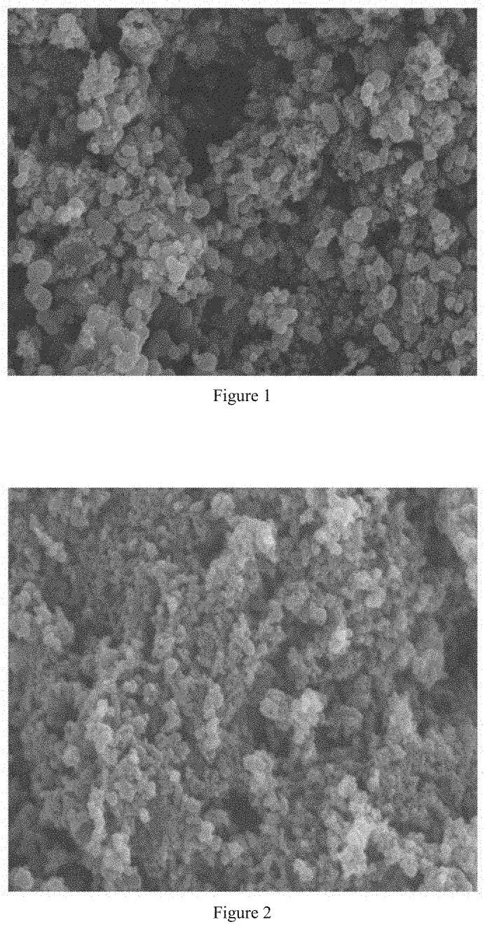

SEM result shows that the obtained sample is microsphere-like and has uniformly distribution, see FIG. 1.

### Example 4

1 g of compound A and 1 g of HPMC-AS were dissolved in 15 ml of N,N-dimethylacetamide. The resulting solution was added dropwise at a rate of 4 g/min into 100 ml of water and stirred. The sample obtained with water as the dispersion medium was used for particle size determination. The resulting suspension was filtered, the suction filtrate appeared milky, and the filter cake was rinsed with water. The resulting solid was dried overnight at 40° C., and subjected to SEM determination.

Particle size of the sample obtained with water as the dispersion medium: D10=7.6 μm, D50=27.2 μm, D90=86 D [4,3]=39.4 μm.

SEM result shows that the obtained sample is granular with uneven particle size, see FIG. 2.

### Example 5

A certain amount of compound A and HPMC-AS-LF were weighed and dissolved in 15 ml of N,N-dimethylacetamide (DMAC). The resulting solution was added dropwise at a rate of 4 g/min into 100 ml of water (adjusted to pH=2 with 36.5% hydrochloric acid) and stirred. The specific parameters are shown in the table below:

| Test | Stirring | Compound | HPMC-AS | pH | Temperature | Stirring rate |
|---|---|---|---|---|---|---|
| Example | method | (g) | (g) | value | (°C) | (rpm) |
| 6 | Mechanical | 1 | 1 | 2 | 25 | 300 |
| | stirring | | | | | |
| 7 | Mechanical | 1 | 1 | 2 | 15 | 300 |
| | stirring | | | | | |
| 8 | Mechanical | 1 | 1 | 2 | 10 | 300 |
| | stirring | | | | | |
| 9 | Mechanical | 1 | 1 | 2 | 5 | 300 |
| | stirring | | | | | |
| 10 | Mechanical | 1 | 1 | 2 | 0 | 300 |
| | stirring | | | | | |

The samples obtained with water as the dispersion medium were subjected to particle size determination respectively. The specific data are as follows:

| Test | D10 | D50 | D90 | D |
|---|---|---|---|---|
| Example | (μm) | (μm) | (μm) | (μm) |
| 6 | 19.9 | 165 | 497 | 223 |
| 7 | 21.6 | 209 | 746 | 349 |
| 8 | 17.5 | 151 | 477 | 203 |
| 9 | 19.3 | 163 | 460 | 204 |
| 10 | 33.1 | 268 | 1430 | 501 |

### Example 6

A solid dispersion comprising compound A and hydroxypropyl methylcellulose acetate succinate was prepared by the method of Test Example 9. A prescription amount of the solid dispersion, lactose, microcrystalline cellulose and croscarmellose sodium were weighed according to the formulation specified as follows. The mixture was poured into a granulating tank, mixed well, and polyvinylpyrrolidone was added as the binder to prepare granules. The wet and soft material was wet-milled and dried, and then the dry granules (water content less than 3%) were dry-milled. Extragranular auxiliary materials were added, and mixed well with the granules. The resulting total mixed granules were compressed into tablets. Specific prescription ratios are shown in Table 2.

TABLE 2

| | Experimental Example | |
|---|---|---|
| | (mg/tablet) | |
| Ingredients | 11 | 12 |
| Compound A | 100 | 100 |
| Hydroxypropyl methylcellulose | 100 | 200 |
| acetate succinate | | |
| Lactose monohydrate | 175 | 175 |
| Microcrystalline cellulose 101 | 60 | 60 |
| Croscarmellose sodium | 25 | 25 |
| (intragranularly) | | |
| Polyvinylpyrrolidone K30 | 20 | 20 |
| Croscarmellose sodium | 15 | 15 |
| (extragranularly) | | |
| Magnesium stearate | 5.0 | 5.6 |
| Total (mg) | 500 | 600 |

Dissolution Test

The dissolution rates of the tablets of Experimental Examples 11 and 12 were determined according to the second method (paddle method) of the dissolution rate test described in general rule of volume IV of Chinese Pharmacopoeia 2015 Edition. The dissolution test was carried out using 1000 ml of 0.15% aqueous solution of SDS as a dissolution medium, at 37±0.5° C., and at a paddle speed of 50 rpm.

TABLE 3

| | Dissolution | |
|---|---|---|
| Time | rate (%) | |
| (min) | 11 | 12 |
| 5 | 60.3 | 63.2 |
| 15 | 80.2 | 82.9 |
| 45 | 97.8 | 98.2 |

1. A method for preparing a solid dispersion, comprising the steps of dissolving a carrier material and the active ingredient (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one or a pharmaceutically acceptable salt thereof in a good solvent to obtain a solution, adding the resulting solution to a poor solvent, and adjusting the pH to 1.0 to 6.0; or, comprising the steps of dissolving a carrier material and the active ingredient in a good solvent to obtain a solution, and adding the resulting solution to a poor solvent, wherein the pH of the poor solvent is 1.0 to 6.0.

2. The method according to claim 1, wherein the good solvent is at least one selected from the group consisting of dimethyl sulfoxide, N,N-dimethylformamide, N,N-dimethylacetamide, acetone, ethanol, tetrahydrofuran and methanol.

3. The method according to claim 1, wherein the poor solvent is at least one selected from the group consisting of diethyl ether, n-hexane, petroleum ether and water.

4. The method according to claim 1, wherein the reagent used to adjust pH is at least one selected from the group consisting of hydrochloric acid, sulfuric acid, acetic acid and phosphoric acid.

5. The method according to claim 1, wherein the addition to the poor solvent is accompanied by a stirring process, the stirring rate is selected from the group consisting of 20 to 1000 rpm.

6. The method according to claim 1, wherein the temperature for the precipitation of solid is selected from the group consisting of 0 to 40° C.

7. The method according to claim 1, comprising:
method 1:
a) dissolving the carrier material and the active ingredient (R)-4-amino-1-(1-(but-2-Preliminary Amendment ynoyl)pyrrolidin-3-yl)-3-(4-(2,6-difluorophenoxy)phenyl)-1,6-dihydro-7H-pyrrolo[2,3-d]pyridazin-7-one or the pharmaceutically acceptable salt thereof in a good solvent, wherein the good solvent is at least one selected from the group consisting of dimethyl sulfoxide, N,N-dimethylformamide, N,N-dimethylacetamide, acetone, ethanol, tetrahydrofuran and methanol,
b) adding the solution obtained in step a) to a poor solvent, and adjusting the pH to 1.0 to 6.0, wherein the poor solvent is at least one selected from the group consisting of diethyl ether, n-hexane, petroleum ether and water;
or, method 2:
a) dissolving the carrier material and the active ingredient (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one or the pharmaceutically acceptable salt thereof in a good solvent, wherein the good solvent is at least one selected from the group consisting of dimethyl sulfoxide, N,N-dimethylformamide, N,N-dimethylacetamide, acetone, ethanol, tetrahydrofuran and methanol,
b) adding the solution obtained in step a) to a poor solvent, wherein the poor solvent is selected from the group consisting of water and a mixed solution of water with one or more of ether, n-hexane and petroleum ether, and the pH of the poor solvent is 1.0 to 6.0.

8. The method according to claim 1, wherein the carrier material is at least one selected from the group consisting of hydroxypropyl methylcellulose acetate succinate, polyvinylpyrrolidone, hydroxypropyl methylcellulose phthalate and polyvinylpyrrolidone.

9. The method according to claim 1, wherein the weight ratio of the carrier material to the active ingredient is 0.5:1 to 4:1, and preferably 0.8:1 to 3.

10. The method according to claim 1, wherein the solid dispersion is consisting of the active ingredient (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one or the pharmaceutically acceptable salt thereof and the carrier material.

11. (canceled)

12. (canceled)

13. (canceled)

14. A method for preparing a solid dispersion, comprising the steps of dissolving a carrier material and an active ingredient in a good solvent to obtain a solution, adding the resulting solution to a poor solvent, and adjusting the pH to 1.0 to 6.0; or, comprising the steps of dissolving a carrier material and an active ingredient in a good solvent to obtain a solution, and adding the resulting solution to a poor solvent, wherein the pH of the poor solvent is 1.0 to 6.0.

15. A solid dispersion comprising the active ingredient (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one or the salt thereof and a carrier material, wherein the particle size D90 of the solid dispersion is 50 μm to 2000 μm.

16. The solid dispersion according to claim 15, wherein the particle size D50 of the solid dispersion is 20 μm to 500 μm.

17. The solid dispersion according to claim 15, wherein the particle size D10 of the solid dispersion is 1 μm to 100 μm.

18. The solid dispersion according to claim 15, wherein the carrier material is at least one selected from the group consisting of hydroxypropyl methylcellulose acetate succinate, polyvinylpyrrolidone, hydroxypropyl methylcellulose phthalate and polyvinylpyrrolidone.

19. The solid dispersion according to claim 15, wherein the weight ratio of the carrier material to the active ingredient is 0.5:1 to 4:1.

20. The solid dispersion according to claim 15, wherein the solid dispersion is consisting of the active ingredient (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one or the pharmaceutically acceptable salt thereof and the carrier material.

21. A solid formulation comprising the solid dispersion prepared by the method according to claim 1 and optionally at least one excipient selected from the group consisting of disintegrant, filler, binder and lubricant.

22. The solid formulation according to claim 21, wherein the disintegrant is at least one selected from the group consisting of croscarmellose sodium, crospovidone, sodium carboxymethyl starch, starch, pregelatinized starch and alginic acid; the binder is at least one selected from the group consisting of polyvinylpyrrolidone, starch, methyl cellulose, carboxy cellulose, hydroxypropyl cellulose, hydroxypropyl methyl cellulose and alginate; and the lubricant is at least one selected from the group consisting of magnesium stearate, stearic acid, palmitic acid, calcium stearate, talc, carnauba wax and sodium stearyl fumarate.

23. The solid formulation according to claim 21, comprising:
1) 10 mg to 500 mg of the active ingredient,
2) 5 to 15% by weight of the disintegrant,
3) 30 to 90% by weight of the filler,
4) 0.5 to 10% by weight of the binder,
5) 0.1 to 5% by weight of the lubricant.

24. The solid formulation according to claim 21, wherein the dissolution rate of the active ingredient determined according to the second method (paddle method) of the dissolution rate test described in general rule of volume IV of Chinese Pharmacopoeia 2015 Edition, using 0.15% aqueous solution of SDS as a dissolution medium, at 37±0.5° C., and at a paddle speed of 50 rpm, is no less than 85%.
